(19) United States
(12) Patent Application Publication
(10) Pub. No.: US 2013/0072794 A1
(43) Pub. Date: Mar. 21, 2013

(54) ULTRASONIC DIAGNOSTIC APPARATUS AND ULTRASONIC TRANSMISSION/RECEPTION METHOD

(71) Applicant: Koji Waki, Tokyo (JP)

(72) Inventor: Koji Waki, Tokyo (JP)

(73) Assignee: HITACHI MEDICAL CORPORATION, Tokyo (JP)

(21) Appl. No.: 13/699,690

(22) PCT Filed: Jun. 1, 2011
(86) PCT No.: PCT/JP2011/062569

§ 371 Date: Nov. 23, 2012

(30) Foreign Application Priority Data
Jun. 4, 2010 (JP) 2010-128618

(51) Int. Cl. A61B 8/08; A61B 8/00; A61B 8/14

(52) U.S. Cl. CPC A61B 8/485; A61B 8/14; A61B 8/5207; A61B 8/54; A61B 8/464; A61B 8/466; A61B 8/4461
USPC 600/438

(57) ABSTRACT

An ultrasonic diagnostic apparatus and ultrasonic transmission/reception method that can acquire high-definition 3D elastic images and are very easy to use. A transmission/reception unit can switch between first transmission/reception condition, under which elasticity frame data is acquired at a first definition, and second transmission/reception condition, under which elasticity frame data is acquired at a second definition which is higher than the first definition. A switching unit detects variations in the elasticity values in the elasticity frame data and switches the aforementioned transmission/reception conditions from the first to the second on the basis of the stability of said variations.

### FIELD OF THE INVENTION

The present invention relates to an ultrasonic diagnostic apparatus that generates and displays an elastic image which shows the distribution of hardness or softness of biological tissue in a region of interest in an object to be examined using ultrasonic waves, in particular to an ultrasonic diagnostic apparatus and ultrasonic transmission/reception method capable of acquiring high-definition 3-dimensional elastic images.

### DESCRIPTION OF RELATED ART

An ultrasonic diagnostic apparatus scans an ultrasonic wave to a tomographic plane (scan surface) in the object by an ultrasonic probe, receives and processes the reflected echo signal reflected from each portion of the biological tissue in the tomographic plane, and acquires the frame data of the RF signal. Then a 2-dimensional or 3-dimensional tomographic image or a 2-dimensional or 3-dimensional elastic image is generated on the basis of the acquired RF signal frame data and displayed on a monitor screen to be used for diagnosis.

Generally, an elastic image is generated based on the two sets of RF signal frame data acquired in the conditions that different amounts of force (compression) are applied to the biological tissue. In other words, using the fact that the biological tissue is deformed according to its hardness due to the applied force, the displacement frame data which shows the displacement in the region of interest or the distribution of the displacement vectors is acquired based on the two sets of RF signal frame data. Then 2-dimensional elastic frame data is generated by obtaining the elasticity value in a measurement point of each portion in the region of interest on the basis of the acquired displacement frame data or the displacement frame data, and a 2-dimensional elastic image is obtained by imaging the generated frame data. Further, by moving the probe to the direction which intersects with the tomographic plane (for example, the orthogonal direction), elastic volume data formed by plural sets of 2-dimensional elastic frame data is acquired. Then a 3-dimensional elastic image is generated by performing rendering using the elastic volume data and the generated image is displayed on a monitor screen.

Various methods are known for creating the condition that different amounts of force are applied to biological tissue. For example, the method which applies pressure or compression to biological tissue via an ultrasonic probe (hereinafter referred to as a probe) using manual or mechanical means, the method using the pressure which is applied to a biological tissue along with the beats of an organ, etc. and the method which applies compression to a biological tissue by the ultrasonic waves irradiated from the probe are known.

Patent Document 1 proposes a technique that consecutively collects plural sets of 2-dimensional elastic frame data by corresponding the data to the variation of compression condition caused by the pressing/releasing of the probe to/from the object and extracts the frame data block having the similar compression from among the collected plural sets of 2-dimensional elastic frame data so as to generate high-definition elastic volume data. In this manner, high-definition 3-dimensional elastic images can be obtained, since the elastic volume data in the similar pressing condition can be acquired even when the pressing condition fluctuates.

### PRIOR ART DOCUMENTS

### Patent Documents

Patent Document 1: JP-A-2008-259555

In ultrasonic diagnosis, generation of 3-dimensional elastic images with improved high-definition has been increasingly demanded. A high-definition 3-dimensional elastic image means in the present specification that the image has high resolution and detailed gradation of elasticity distribution. In order to generate such high-definition 3-dimensional elastic images, high resolution and high frame rate are required in plural sets of 2-dimensional elastic frame data for constructing elastic volume data. However, in order to acquire such high-definition 2-dimensional elastic frame data, stability and uniformity is required in pressing and releasing operation of the probe. Also in addition to the high frame rate, collection of elastic frame data having dense frame intervals is required by moving the probe in the direction which intersects with the tomographic plane (scanning plane) as slowly and evenly as possible. On the other hand, when a target region of interest (diagnostic target region) is searched while observing a high-definition 2-dimensional elastic image, it takes time for searching and prolongs the time for collecting high-definition elastic volume data of a desired region of interest, thereby lowering the availability of the ultrasonic diagnostic apparatus.

Given this factor, in order to search for a region of interest, it is preferable to obtain a 2-dimensional tomographic image (or a 2-dimensional elastic image) in a search mode with a rough resolution and low frame rate, then when the region of interest is captured within the image, obtain a 2-dimensional tomographic image (or a 2-dimensional elastic image) in a high-definition mode with a high resolution and high frame rate.

In the conventional technique disclosed in Patent Document 1, there is no consideration regarding generation of high-definition 3-dimensional elastic images by switching a searching mode and a high-definition mode of a region of interest for promptly searching the region of interest and collecting high-definition elastic volume data.

The objective of the present invention is to provide the ultrasonic diagnostic apparatus and ultrasonic transmission/reception method with improved availability capable of obtaining high-definition 3-dimensional elastic images.

### BRIEF SUMMARY OF THE INVENTION

In order to achieve the above-described objective, the ultrasonic diagnostic apparatus of the present invention comprises:

a transmission/reception processing unit configured to scan ultrasonic beams to an object to be examined via a probe and receives the ultrasonic signals from the object;

a 2-dimensional elastic image constructing unit configured to generate a 2-dimensional elastic image by acquiring elastic frame data showing the distribution of elasticity values on the basis of the ultrasonic signals;

a 3-dimensional elastic image constructing unit configured to generate a 3-dimensional elastic image on the basis of the plural sets of elastic frame data; and

a display unit configured to display at least one of the 2-dimensional elastic image and the 3-dimensional elastic image,

characterized in further comprising:

a switching unit configured to detect variation of elasticity value in the plural sets of elastic frame data and switch the transmission/reception condition of the transmission/reception processing unit on the basis of the stability in the variation of the elasticity value.

In other words, if the variation of elasticity value in plural sets of elastic frame data is stabilized, appropriate elastic frame data can be acquired in any method of applying pressure to biological tissue using beats or ultrasonic waves, etc. by manual or mechanical means. Given this factor, by switching the transmitting/receiving condition of the transmission/reception unit to a condition, for example capable of obtaining a high-definition 3-dimensional image when the stability is detected in variation of the elasticity value, it is possible to provide the ultrasonic diagnostic apparatus with improved usability capable of automatically obtaining high-definition 3-dimensional elastic images.

In this case, it is preferable that the transmitting/receiving condition is set as a first transmitting/receiving condition which acquires the elastic frame data with the first definition and a second transmitting/receiving condition which acquires the elastic frame data with the second definition which is higher than the first definition. Then the switching unit downloads the plural consecutive sets of elastic frame data that are acquired with the first transmitting/receiving condition from the 2-dimensional elastic image constructing unit and evaluates the stability in variation of the elasticity value.

In other words, an examiner sets the first transmitting/receiving condition with the rough first definition on the transmission/reception processing unit, transmits/receives ultrasonic waves by applying a probe on the body surface of the object, and causes the display unit to display a 2-dimensional elastic image (and/or a 2-dimensional tomographic image) of the first definition. At this time, since the 2-dimensional elastic images indifferent tomographic plane are sequentially displayed on the display unit, the examiner can search a desired region of interest by moving the probe in the direction that intersects with the tomographic plane (for example, the direction which is approximately orthogonal) while observing the 2-dimensional elastic images. Here, by setting the rough first definition, the measurement time of 2-dimensional elastic frame data can be shortened. Also by setting the rough frame rate, the probe can be moved quickly thereby the time for searching a desired region of interest can be reduced. In addition, while the search of a region of interest can be performed using 2-dimensional tomographic images, since the start of acquiring elastic volume data is to be automatically detected based on the variation of the elasticity value as will be described later, the 2-dimensional elastic images are to be generated and displayed even in the case of the search mode.

Here, the operation to move the probe in the direction that intersects with the tomographic plane can be executed manually. The mechanical operation can be executed, for example by mounting a motor-driving jig which makes the probe swing in the direction intersecting with the tomographic plane. Further, a 2-dimensional arrayed electric scanning probe in which transducers are 2-dimensionally arrayed can be used to make an ultrasonic beam swing in the direction that intersects with the tomographic plane by electric scanning.

In the case that an examiner starts acquisition of a 3-dimensional elastic image at the position where a region of interest is searched as described above, for example by pressing and releasing the probe applied on the body surface of an object, a 2-dimensional elastic image of the region of interest with the first definition is displayed on the display unit. The elasticity value to be displayed on the 2-dimensional elastic image varies in accordance with the pressing and releasing operation of the probe. Given this factor, the switching unit obtains the variation pattern of the elasticity value in the 2-dimensional elastic image with the first definition, and the examiner can determine that the acquisition of a high-definition 3-dimensional elastic image has started if the obtained variation pattern is stable. The high-definition elastic volume data can be acquired by outputting the command to switch the transmitting/receiving condition to the second definition to the transmission/reception unit on the basis of the previously mentioned determination, thereby a high-definition 3-dimensional elastic image can be obtained.

In other words, the switching command which is output from the switching unit is the trigger signal for starting acquisition of a high-definition elastic image. In this manner, in accordance with the present invention, a region of interest can be searched with high speed using a rough-definition ultrasonic image (a 2-dimensional elastic image and/or a 2-dimensional elastic image). Then when acquisition of the elastic volume data is started by the examiner at the position where the region of interest is captured, variation of the elasticity value appears in the 2-dimensional elastic frame data. Given this factor, it is possible to provide an ultrasonic diagnostic apparatus with improved availability by automatic switching performed by the switching unit to the acquisition of high-definition elastic volume data on the basis of the variation in the elasticity value. In other words, a proper timing for switching can be obtained automatically to acquire high-definition elastic frame data from the variation in the elasticity value such as the displacement detected from an ultrasonic image, whereby improving simplicity of the operation and enabling acquisition of high-definition 3-dimensional images.

While an example of performing manual operation to apply pressure on biological tissue of an object is described above for obtaining elastic images, the present invention is not limited to this case. For example, in the case that the probe applied to the body surface of the object is mechanically pressed and released, by operating a switch of a swinging jig to which a probe is attached, the mechanical pressing/releasing operation appears in the variation of the elasticity value. Given this factor, it is possible to automatically switch to the high-definition mode by detecting the start of mechanical pressing/releasing operation based on the variation pattern in the elasticity value. Also in the same manner, in the case that the pressure applied to biological tissue caused by beats is used or the case that the pressure is applied to biological tissue using the ultrasonic waves irradiated from the probe, since the searching speed in the search mode varies irregularly, the interval (pitch) between two sets of RF frame data also varies irregularly. Therefore, since the variation pattern of the elasticity value fluctuates irregularly, it is possible to automatically switch to the high-definition mode by detecting that the variation of elasticity value is changed to a regular and stable one. In this manner in any pressing method, switching to the high-definition mode can be performed automatically by detecting the start of acquiring elastic volume data when the variation pattern of the elasticity value is stabilized.

The elasticity frame data can be calculated on the basis of the RF signal frame data acquired by the first or the second definition respectively. Also, the first or the second definition can be set using the density of the transmission/reception beam and/or the frame rate. Also, any of the displacement in biological tissue, strain, elasticity modulus, viscosity, strain ratio with respect to a reference region, and other physical quantities (parameters) which correlate with elasticity can be used as the elasticity value.

In any of the above-described ultrasonic diagnostic apparatuses, the stability of variation in the elasticity value can be evaluated based on the detected variation pattern of the elasticity value. The stability in variation pattern of an elasticity value can be evaluated, when there is a cycle that the variation pattern repeatedly increases and decreases, based on the variation pattern feature quantity of two consecutive half a cycles or a cycle of the variation cycle. For example, the difference between two temporally consecutive variation patterns is acquired, and the variation pattern is evaluated as stable if the acquired difference is within a set range. Also, the switching unit, in addition to the stability of variation cycles, switches the transmitting/receiving condition from the first definition to the second definition based on the continuity of stability for the set number of cycles.

Further, in any of the above-described ultrasonic diagnostic apparatuses, the 2-dimensional elastic image or the 3-dimensional elastic image with the first definition can be displayed on the display unit.

Furthermore, in any of the above-described ultrasonic diagnostic apparatuses, the 3-dimensional elastic image constructing unit can construct a 3-dimensional elastic image by dividing plural sets of elastic volume data into the plural frame blocks respectively, creating one set of elastic volume data by combining the frame blocks having the elasticity value within a certain allowable range, and rendering the combined elastic volume data. Accordingly, a 3-dimensional elastic image can be constructed using the block of elastic volume data having appropriate elasticity value, whereby the 3-dimensional elastic image can be constructed with further improved high-definition.

Any of the above-described switching units, when the variation pattern becomes unstable after switching to the second transmitting/receiving condition, can reset the first transmitting/receiving condition, then switch to the second transmitting/receiving condition again based on the evaluation of variation pattern stability.

Any of the above-described 2-dimensional elastic image constructing units acquires the elastic frame data showing the distribution of the elasticity value in biological tissue that deforms by receiving pressure caused by beats, and the switching unit can load the consecutive plural sets of elastic frame data, detect the peak of the variation pattern in the elasticity value caused by the beats and switch the transmitting/receiving condition of the transmission/reception unit based on the stability in the peak cycle. In this case, when the probe is attached to the jig which by a motor makes the probe swing in the direction which intersects with the tomographic plane, the switching unit can output the signal to stop the motor for a set period of time upon detecting the peak, and after passing of a set period of time, drive the motor again so that the tomographic plane position of the probe is made to swing at a set angle in accordance with the peak cycle.

Also, in any of the above-described ultrasonic diagnostic apparatuses, the 3-dimensional elastic image constructing unit can construct a 3-dimensional elastic image by respectively dividing plural sets of elastic volume data into plural frame blocks, creating one set of elastic volume data by combining the frame blocks having the elasticity value within a certain allowable range, and rendering the combined elastic volume data.

The stability of variation in the elastic value can be evaluated by comparing the correlation value or the noise ratio between the two sets of adjacent elastic frame data which form the elastic volume data acquired by the first transmitting/receiving condition and a set value thereof. In this case, it is preferable to evaluate the stability based on the correlation value of two sets of adjacent elastic frame data or the average value of the noise ratio in the whole volume data. Further, the stability of variation in the elastic value can be evaluated by loading the plural sets of elastic volume data acquired by the first transmitting/receiving condition and referring to the degree of similarity in the two consecutive sets of elastic volume data.

### EFFECT OF THE INVENTION

In accordance with the present invention, it is possible to provide the ultrasonic diagnostic apparatus with improved availability capable of obtaining high-definition 3-dimensional elastic images.

### BRIEF DESCRIPTION OF THE DIAGRAMS

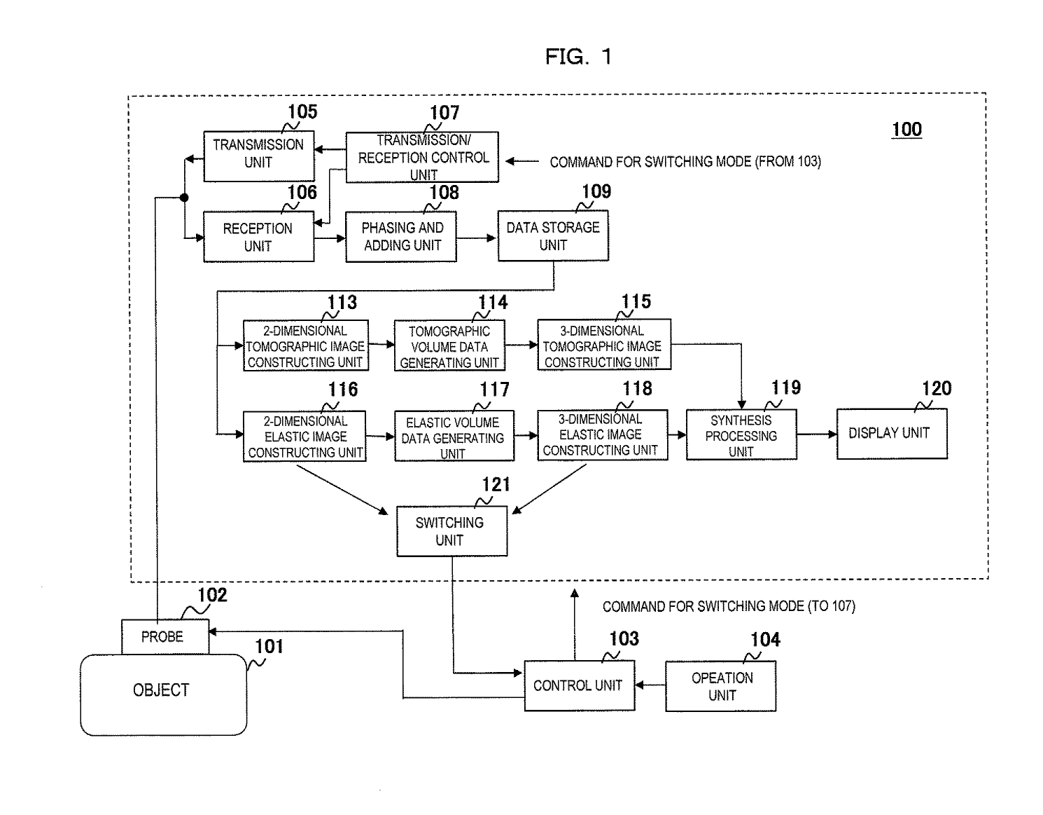

FIG. 1 is a block configuration diagram showing the general configuration of an embodiment in the ultrasonic diagnostic apparatus related to the present invention.

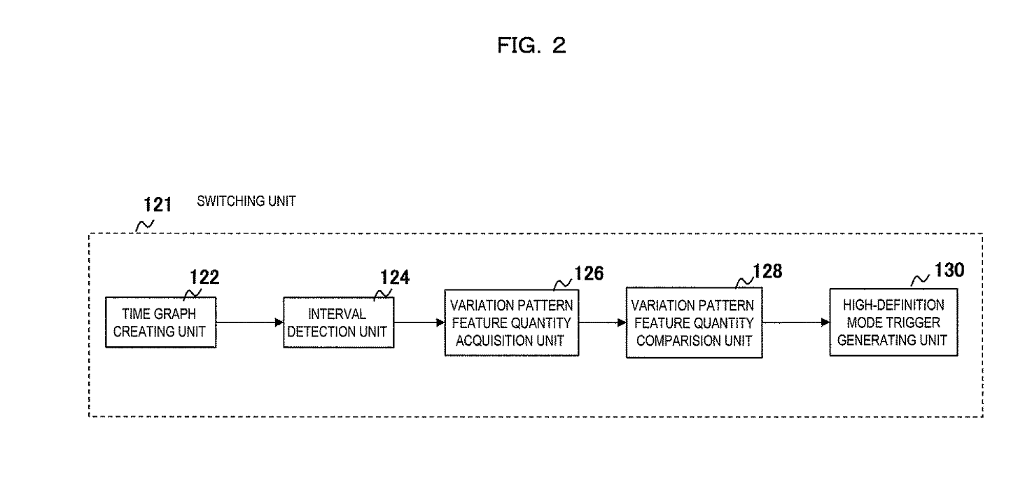

FIG. 2 is a detailed block configuration diagram of a switching unit in FIG. 1.

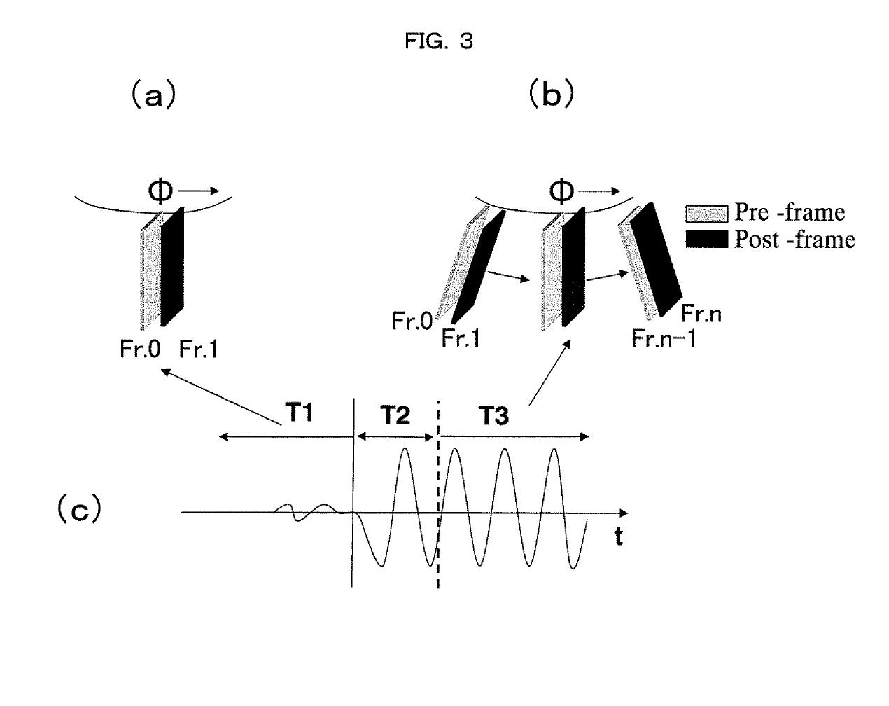

FIG. 3 is a view for explaining the operation of a switching unit in a first embodiment.

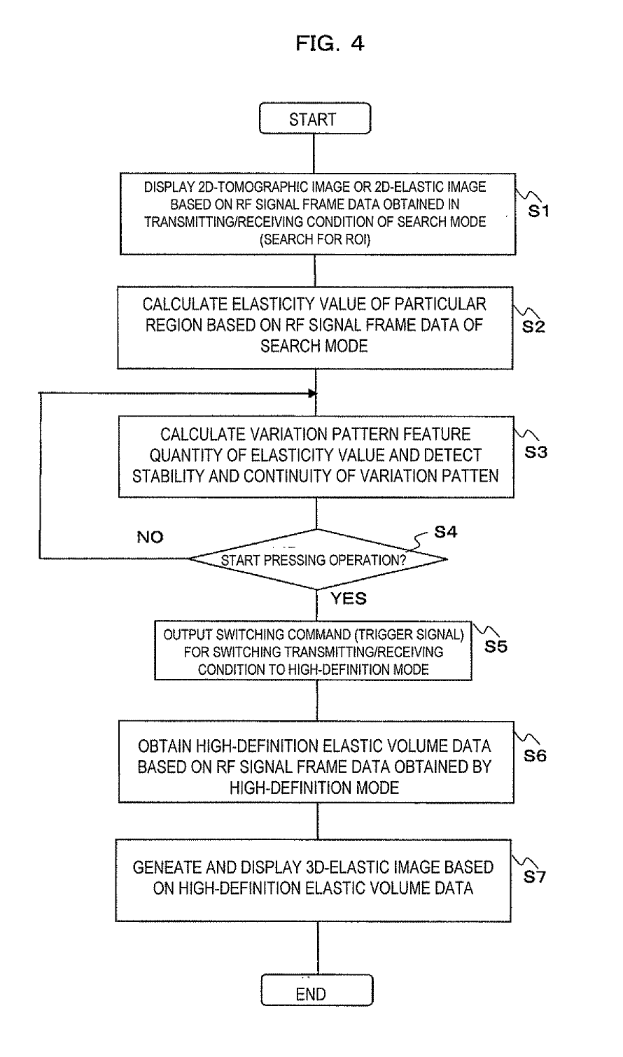

FIG. 4 is a flowchart showing the general operation of an embodiment in the ultrasonic diagnostic apparatus related to the present invention.

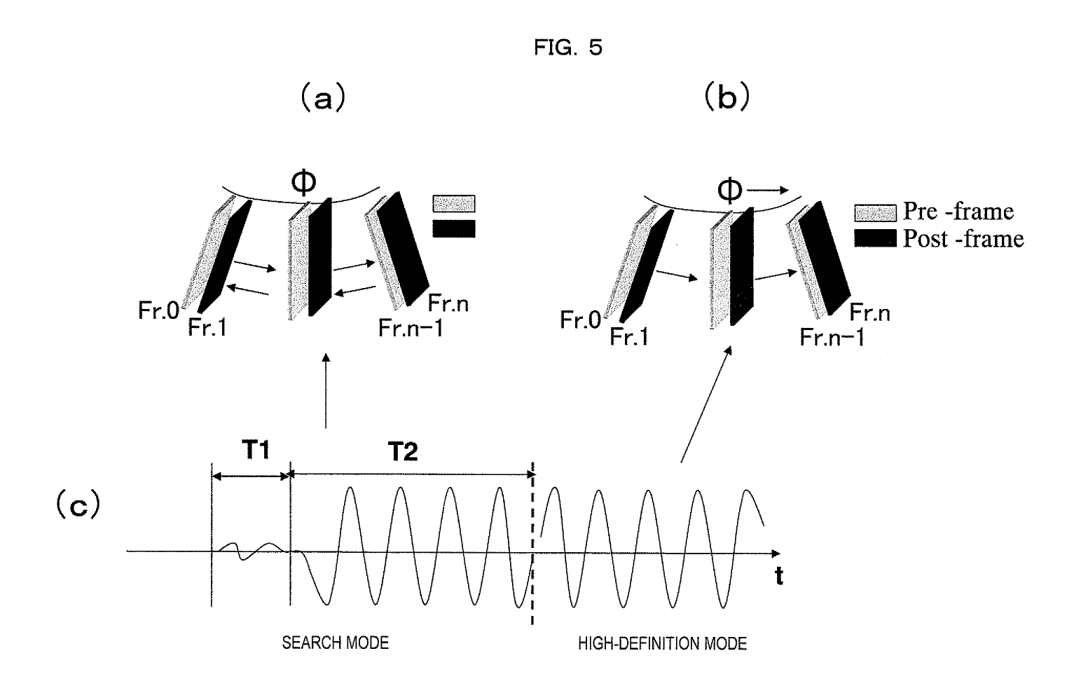

FIG. 5 is a view for explaining the operation of a switching unit in a modification of the first embodiment.

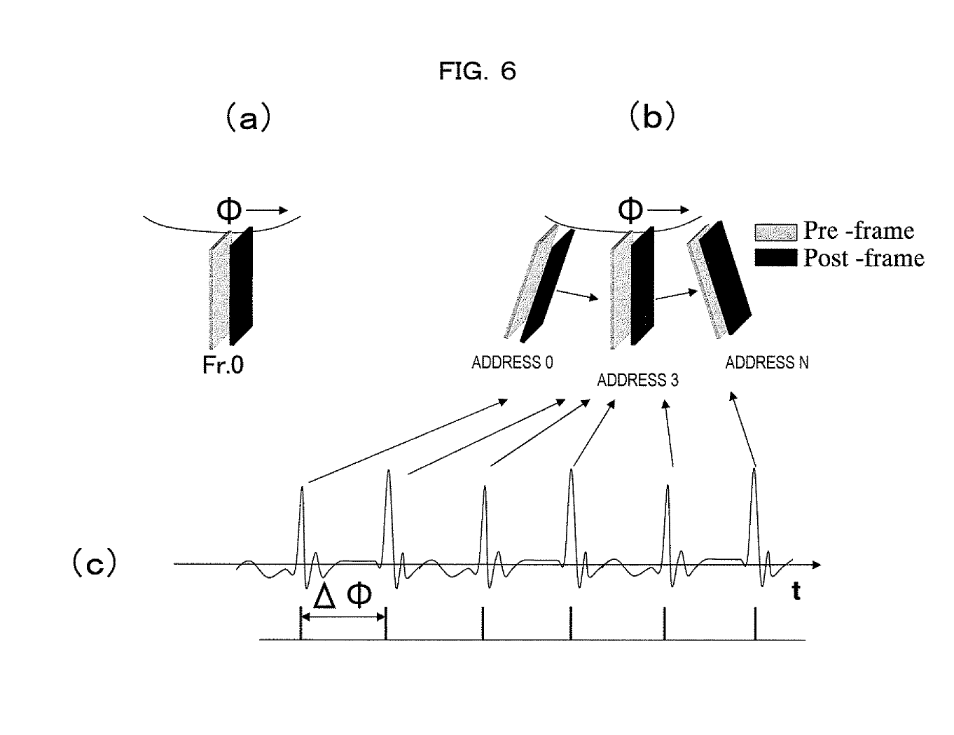

FIG. 6 is a view for explaining the operation of the switching unit in a second embodiment.

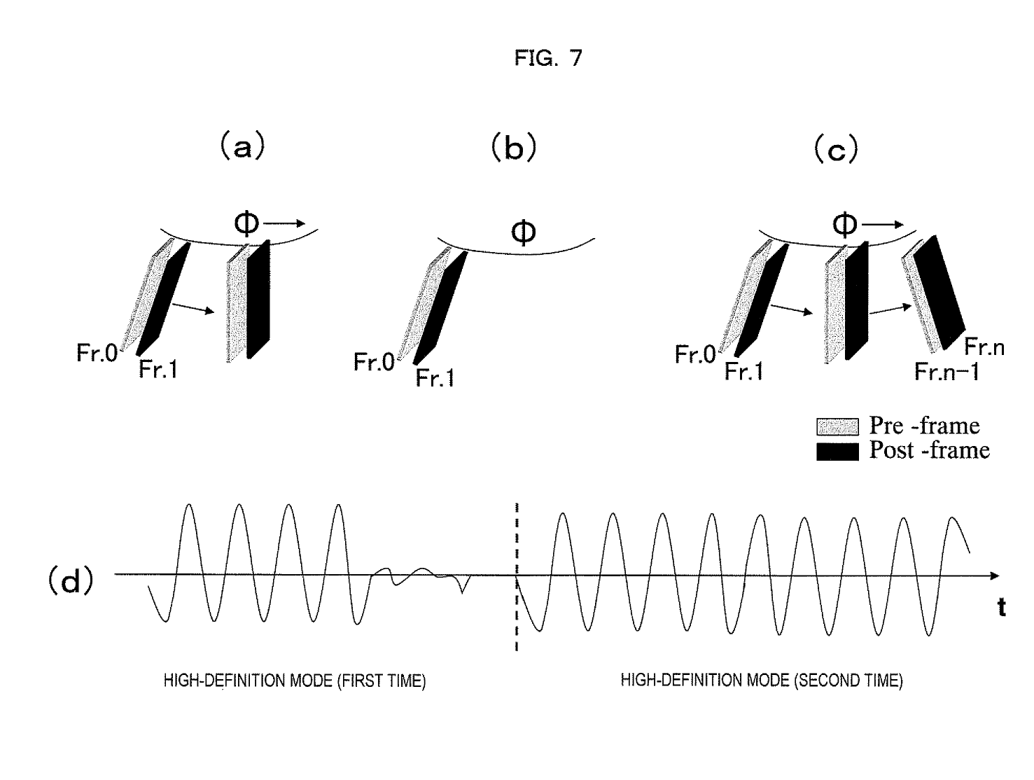

FIG. 7 is a view for explaining the operation of the switching unit in a third embodiment.

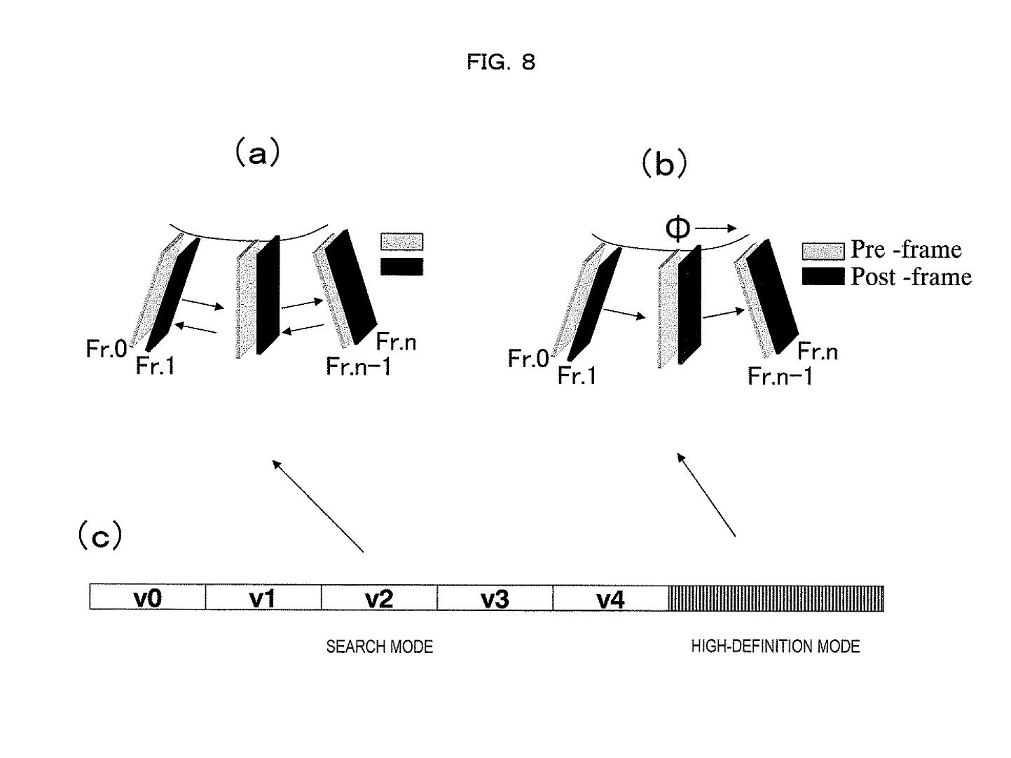

FIG. 8 is a view for explaining the operation of the switching unit in a fourth embodiment.

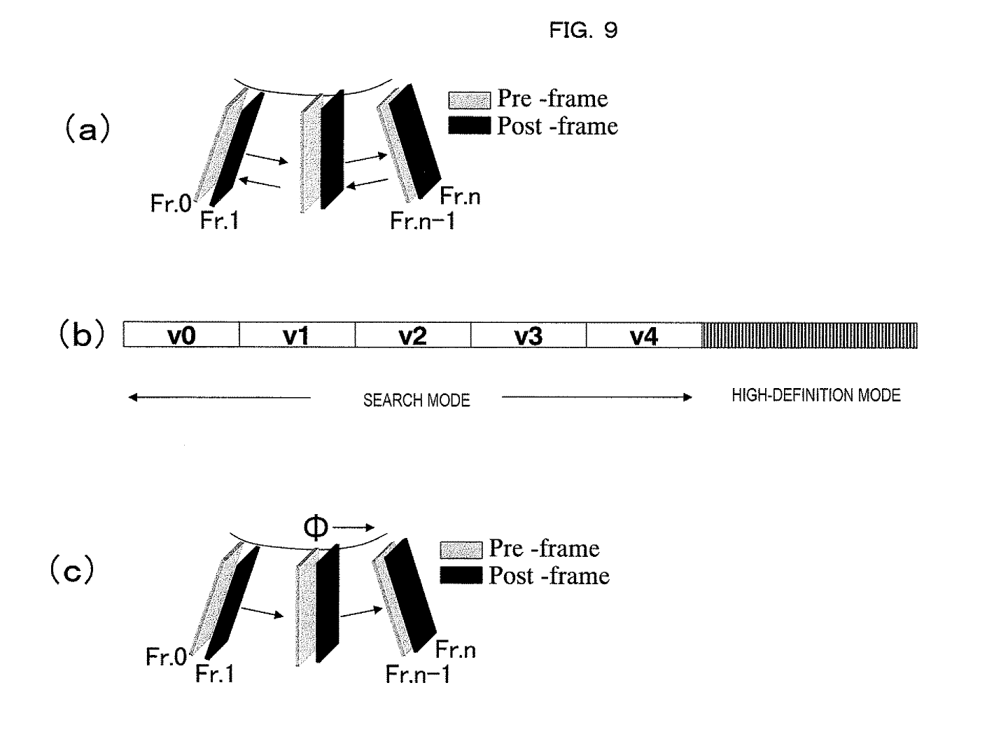

FIG. 9 is a view for explaining the operation of the switching unit in a fifth embodiment.

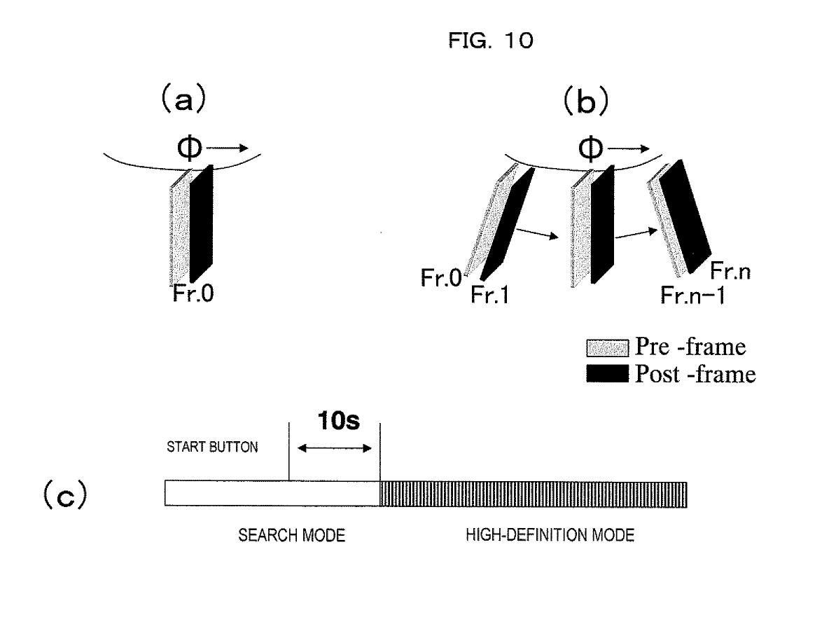

FIG. 10 is a view for explaining the operation of the switching unit in a sixth embodiment.

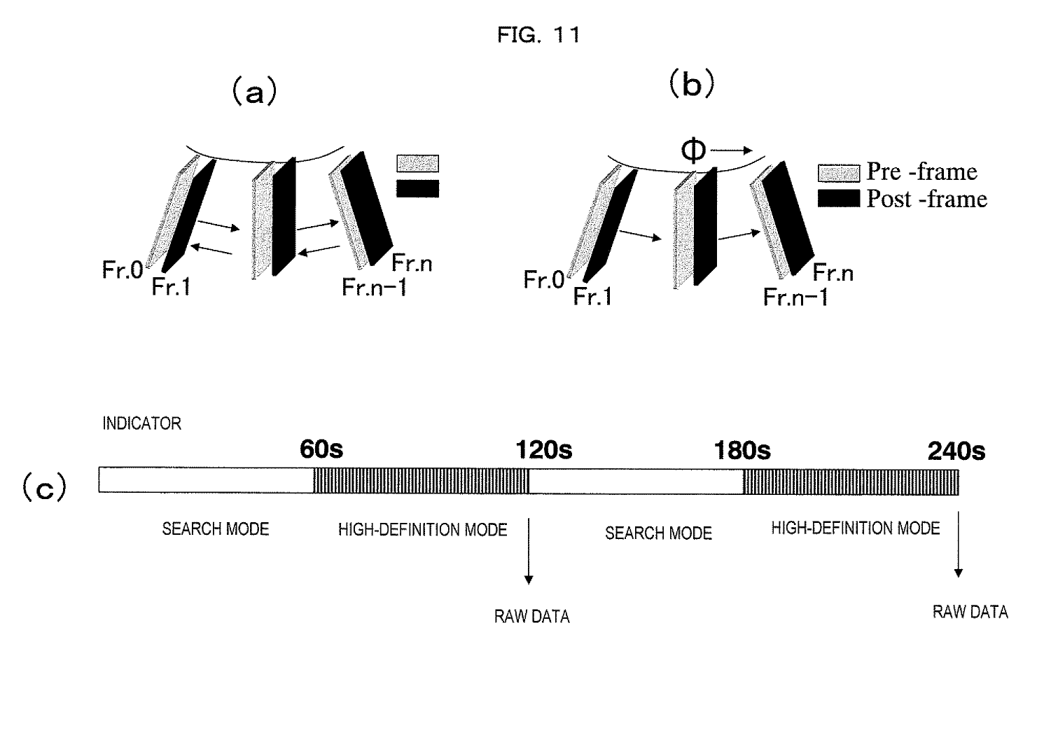

FIG. 11 is a view for explaining the operation of the switching unit in a seventh embodiment.

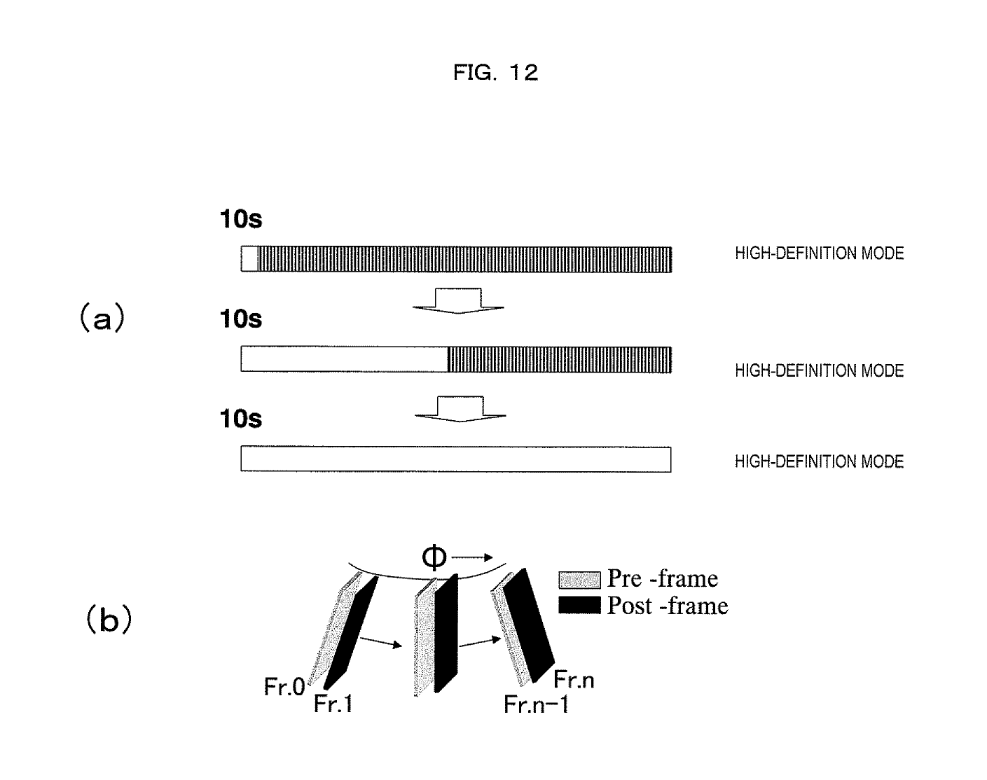

FIG. 12 is a view for explaining an example of a progress bar in the seventh embodiment.

### DETAILED DESCRIPTION OF THE INVENTION

FIG. 1 shows a block configuration diagram showing the general configuration of an embodiment in the ultrasonic diagnostic apparatus related to the present invention. As shown in the diagram, the ultrasonic diagnostic apparatus comprises a main body 100, an ultrasonic probe (hereinafter abbreviated as a probe) 102 to be used by applying on the body surface of an object 101, a control unit 103 configured to control the respective components of the main body 100, and an operation unit 104 for inputting various commands to the control unit 103. The operation unit 104 comprises a pointing device such as a keyboard or a trackball. The probe 102 is provided with plural transducers arrayed in a rectangle or fan shape, configured to transmit/receive ultrasonic waves via the transducers to/from the object 101. In the present embodiment, it is configured so that the probe 102 is made swung mechanically in the direction orthogonal to the array direction of the transducers (the short-axis direction) for transmitting and receiving the ultrasonic waves to a 3-dimensional region. For example, by attaching the probe 102 to a jig which makes the probe 102 swing in the direction that intersects with the tomographic plane (for example, orthogonal), so as to make the probe 102 swing by a motor which is mounted in the jig. This type of probe is a so-called mechanical 3-dimensional probe. In addition, instead of mechanically swinging the probe 102, the present invention can be performed by manually swinging the probe 102. Also, ultrasonic waves may be transmitted and received to/from a 3-dimensional region by electronically controlling the transmission and reception using the probe 102 in which plural transducers are arrayed 2-dimensionally.

The main body 100 of the ultrasonic diagnostic apparatus comprises a transmission unit 105 configured to repeatedly transmit ultrasonic waves to a tomographic plane of the object 101 via the probe 102 at predetermined time intervals, a reception unit 106 configured to receive the reflected echo signals from the biological tissue corresponding to the ultrasonic waves transmitted to the object 101 via the probe 102, and a transmission/reception control unit 107 configured to control the transmission unit 105 and the reception unit 106.

The transmission unit 105 generates a transmission pulse for generating an ultrasonic wave by driving the transducers of the probe 102. The transmission unit 105 has a function to set the convergent point of the transmitted ultrasonic waves at a certain depth. Also, the reception unit 106 generates an RF signal, i.e. reception signal by amplifying the reflected echo signal received by the probe 102 at a predetermined gain. The transmission/reception control unit 107 controls the transmission unit 105 and the reception unit 106. The phasing and adding unit 108 controls the phase of the RF signal amplified in the reception unit 106, and generates the wave-receiving beam of the ultrasonic waves with respect to one or plural convergent points. The RF signal of the wave-receiving beam output from the phasing and adding unit 108 is stored in a data storage unit 109 as the RF signal frame data corresponding to the tomographic plane.

The RF signal frame data stored in the data storage unit 109 is sequentially loaded in a 2-dimensional tomographic image constructing unit 113, and 2-dimensional tomographic frame data is generated. The 2-dimensional tomographic image constructing unit 113 inputs the RF signal frame data output from the data storage unit 109 on the basis of the condition set by the control unit 103, executes signal processing such as gain compensation, log compression, detection, edge enhancement and filtering, and generates 2-dimensional tomographic frame data. The 2-dimensional tomographic frame data generated in the 2-dimensional tomographic image constructing unit 113 is output to a tomographic volume data generating unit 114. The tomographic volume data generating unit 114 generates 3-dimensional tomographic volume data by appending the 3-dimensional spatial coordinates to sequentially input plural sets of 2-dimensional tomographic frame data, and stores the generated data in a memory. A commonly-known method can be applied for appending 3-dimensional spatial coordinates to 2-dimensional tomographic frame data. For example, the probe 102 is configured capable of measuring the transmitting/receiving direction (θ,φ) at the same time as transmitting/receiving ultrasonic waves. Here, θ is a scan angle of the ultrasonic beam which scans in a fan shape along the tomographic plane, and φ is a swinging angle of the RF signal frame to be swung in the direction that intersects with the tomographic plane. The tomographic volume data generating unit 114 performs 3-dimensional coordinate conversion on the plural sets of 2-dimensional tomographic frame data on the basis transmitting/receiving directions (θ,φ) equivalent to the positions where the 2-dimensional tomographic frame data is acquired, and generates the tomographic volume data.

The 3-dimensional tomographic image data constructing unit 115 is configured to generate 3-dimensional tomographic frame data using a commonly-known rendering means to be described below based on the luminance and opacity of the 3-dimensional tomographic volume data generated by the tomographic volume data generating unit 114. More specifically, the rendering is performed using the following equations (1)˜(3).

Cout(i)=Cout(i−1)+(1−Aout(i−1))·A(i)·C(i)·S(i) (1)

Aout(i)=Aout(i−1)+(1−Aout(i−1))·A(i) (2)

A(i)=Opacity[C(i)] (3)

C(i) is, when a 3-dimensional tomographic image is viewed from a certain point on the created 2-dimensional projection surface, the luminance value of the i-th voxel exists in the line of sight. Cout (i) is the output pixel value. For example, when the luminance values of N voxels are aligned in the line of sight, the luminance value Cout (N−1) in which up to i=0˜(N−1) are integrated becomes the pixel value to be ultimately output. Cout(i−1) indicates the integrated value up to the (i−1)-th voxel.

Also, A(i) is the opacity of the i-th luminance value exists in the line of sight, and is a tomographic opacity table in the values of 0˜0.1 as shown in the equation (3). The tomographic opacity table determines the contribution rate on the output 2-dimensional projection surface (3-dimensional tomographic image) by referring to the opacity from the luminance value.

S(i) is the weighting element for shading which is calculated by the gradient acquired by the luminance C(i) and the surrounding pixels, and indicates the accentuation effect that, for example, 1.0 is given for maximum reflection when the luminous source coincides with the normal line on the surface centering around voxel i and 0.0 is given when the luminous source and the normal line are orthogonal to each other.

Both Cout(i) and Aout(i) have 0 as the initial value. As shown in the equation (2), Aout (i) is integrated each time of passing a voxel and converged to 1.0. Thus as shown in the equation (1), when the integrated value Aout(i−1) of opacity up to the (i−1)th voxel is about 1.0, the luminance value C(i) after the i-th voxel will not be reflected on the output images.

On the other hand, the RF signal frame data stored in the data storage unit 109 is sequentially loaded in the 2-dimensional elastic image constructing unit 116, and 2-dimensional elastic image data is generated. In other words, the 2-dimensional elastic image constructing unit 116 acquires the displacement of the respective portions in a region of interest based on the plural sets of RF signal frame data stored in the storage unit 109 acquired at different times, i.e. having different pressing conditions.

Then the elasticity value is calculated on the basis of the acquired displacement, and 2-dimensional elastic frame data is generated. Here, any one of the displacement, strain, elasticity modulus, viscosity, strain ratio with respect to a set reference region and the other physical quantity (parameter) to be correlated with elasticity can be used as the elasticity value. The plural sets of 2-dimensional elastic frame data that are sequentially generated in the 2-dimensional elastic image constructing unit 116 are output to the elastic volume data generating unit 117. The elastic volume data generating unit 117 appends the 3-dimensional spatial coordinates on the sequentially input plural 2-dimensional elastic frame data to generate the 3-dimensional elastic volume data, and stores the generated data in a memory. The method of appending 3-dimensional spatial coordinates to 2-dimensional elastic frame data is the same as the method used in the case of the above-described 2-dimensional tomographic frame data

As in the case of the above-described 3-dimensional tomographic images, the 3-dimensional elastic image constructing unit 118 generates 3-dimensional elastic images based on the 3-dimensional elastic volume data generated by the elastic volume data generating unit 117 using a commonly-known rendering means. At this time, as disclosed in Patent document 1, the 3-dimensional elastic image constructing unit 118 is capable of dividing the plural sets of elastic volume data in which a region of interest is imaged respectively into plural frames, creating one set of elastic volume data by combining the frame blocks having the elasticity value within a certain allowable range and constructing a 3-dimensional elastic image by rendering the created elastic volume data. In this manner, since a 3-dimensional elastic image can be constructed from the blocks of elastic volume data having appropriate elasticity values, it is possible to construct a 3-dimensional elastic image with improved high-definition.

The 3-dimensional tomographic frame data generated in the 3-dimensional tomographic image constructing unit 115 and the 3-dimensional elastic frame data generated in the 3-dimensional elastic image constructing unit 118 are arbitrarily loaded to a synthesis processing unit 119 in accordance with the command input from the operation unit 104 via the control unit 103 or the control unit 103. The synthesis processing unit 119 juxtaposes a 3-dimensional tomographic image and a 3-dimensional elastic image according to the command input from a device such as the control unit 103 or generates a composite image processed by additive synthesis, etc., and displays the generated images on the display unit 120.

Here, the characteristics of the present embodiment will be described. The first characteristic is in the transmission/reception processing unit which is formed by the transmission unit 105, the reception unit 106 and the transmission/reception control unit 107. The transmission/reception unit is configured swichable between the first transmitting/receiving condition for acquiring 2-dimensional tomographic frame data and 2-dimensional elastic frame data with the preset first definition and the second transmitting/receiving condition for acquiring 2-dimensional tomographic frame data and 2-dimensional elastic frame data with the second definition which is higher than the first definition. Also, the second characteristic is that a switching unit 121 is provided.

The switching unit 121 loads the plural sets of 2-dimensional elastic frame data acquired with the first transmitting/receiving condition that are sequentially generated in the 2-dimensional elastic image constructing unit 116, and detects the variation of the elasticity value in the 2-dimensional elastic frame data. Then based on the variation pattern of the elasticity value, i.e. when the variation pattern is detected which appears that the examiner has started acquisition of elastic volume data by the high-definition mode, the switching unit outputs to the control unit 103 a high-definition mode switching command for switching the condition in the transmission/reception processing unit to the second transmitting/receiving condition. The control unit 103 is configured to control the transmission/reception control unit 107 to switch the transmitting/receiving condition of the transmission/reception processing unit from the first condition to the second condition based on the high-definition mode switching command. The detailed configuration and the operation of the switching unit 121 will be described below on the basis of embodiments. The transmission/reception processing unit is formed at least by the transmission unit 105 and the reception unit 106.

### Embodiment 1

FIG. 2 shows the detailed configuration of the switching unit 121 in an embodiment. The switching unit 121 automatically detects the start of acquisition of high-definition elastic volume data based on the variation of the elasticity value, and outputs the switching command which is a trigger signal to the high-definition mode. More specifically, the switching unit 121 is configured by a time graph creating unit 122, an interval detecting unit 124, a variation pattern feature quantity acquisition unit 126, a variation pattern feature quantity comparison unit 128 and a high-definition mode trigger generation unit 130.

The time graph creating unit 122 temporally stores the information such as an elasticity value (strain, elasticity modulus, displacement, viscosity or strain ratio) and pressure which is acquired in the 2-dimensional elastic image constructing unit 116, and executes display of the stored information. In the present embodiment, a search of a region of interest is executed by moving the probe 102 with an arbitrary search speed and a fixed swinging angle φ while acquiring two sets of RF signal frame data Fr.0 and Fr.1 with the first definition frame rate and observing the 2-dimensional elastic image displayed on the display unit 120. At this time, the 2-dimensional elastic image is obtained by manually pressing and releasing the probe 102 to and from the object 101 (hereinafter arbitrarily referred to as pressing operation). In this manner, the elasticity value of the biological tissue in accordance with the intensity and cycles of the pressure applied to the biological tissue is reflected to the time graph. FIG. 3(3) shows an example of the time graph.

FIG. 3(c) shows an example which uses displacement as an elasticity value. The displacement can be detected, as known in the art, by local tracking or autocorrelation between two frames based on a pair of RF frame data sets Fr.0 and Fr.1 measured at different times as shown in FIG. 3(a).

While the time graph may be created based on the information such as the average value of the displacement in the entire 2-dimensional elastic frame data, it is preferable to create a time graph based on the displacement between two local points in a specified section such as a fat layer or a displacement image of a specified section such as a fat layer. In this manner, not only the time for creating the graph can be reduced but also the time graph of displacement can be obtained stably.

In time zone T1 of the search mode for searching a region of interest, the transmission/reception control unit 107 controls the transmission unit 105 and the reception unit 106 with the first transmitting/receiving condition of the rough definition which is set corresponding to the search mode. For example, the first transmitting/receiving condition is set with rough density in the number of ultrasonic beams for scanning on the tomographic plane and a low frame rate (i.e. low volume rate). Then the examiner searches the region of interest while observing the 2-dimensional tomographic image or the 2-dimensional elastic image displayed on the display unit 120 and the swinging angle φ of the probe 102 is being fixed as shown in FIG. 3(a). Since no conscious pressing operation is performed during the search mode, as shown in time zone T1 of FIG. 3(c), the detected displacement is relatively small. When the region of interest is captured, in the time zone T2, the examiner attempts to start acquiring elastic volume data by performing conscious pressing operation. In this manner, in the time zone T2 of FIG. 3(c), large variation is recognized periodically in the displacement according to the pressing and releasing operation of the probe 102.

The interval detection unit 124 loads the displacement graph which is created in the time graph creation unit 122, and divides the variation cycle of the displacement into every half cycle. In this interval division, the zero-crossover point of the variation cycle can be the halfway mark of an interval by taking the center of the displacement width in the pressing and releasing operation as the benchmark (zero). However, the variation cycle can also be divided into every half a cycle which is the switching point of the pressing and releasing operation and is sandwiched between the maximum point and the minimum point of the displacement. The variation pattern feature quantity acquisition unit 126 calculates the variation pattern feature quantity of the displacement for every half-a-cycle divided in the interval detection unit 124, and outputs the calculated quantity to the variation pattern feature quantity comparison unit 128. Here, the variation pattern feature quantity is the quantity capable of presenting the property of the pattern (form) in the variation cycles. It is the pattern feature quantity capable of determine the degree of approximation, uniformity and so on between the two variation patterns wherein the variation cycle is segmented by half-a-cycle, to which a value such as the average value or average deviation can be applied. Here, the average deviation is the degree of fluctuation in the measurement value, which is the square root of the value in which the absolute value of the displacement in the respective measurement points of the time axis is divided by the average value X the number of measurement points thereof.

The variation pattern feature quantity comparison unit 128 sequentially acquires the difference between two consecutive variation pattern feature quantities as an evaluation parameter. Then the variation pattern feature quantity comparison unit 128 compares the evaluation parameter with a predetermined evaluation range, evaluates the compared result as “stable” if the parameter is within the evaluation range, and outputs the high-definition mode switching command (trigger signal) via the high-definition mode trigger generation unit 130. In this case, the variation pattern feature quantity comparison unit 128 can further detect that the variation pattern in which the evaluation parameter is evaluated as “stable” continued for predetermined plural intervals, evaluate the variation pattern as “successive”, and add continuity of variation pattern feature quantity to stability as the condition for outputting the high-definition mode switching command.

When the high-definition mode switching command is output from the high-definition mode trigger generation unit 130, the command to switch the transmission/reception condition is output to the transmission/reception control unit 107 via the control unit 103. The transmission/reception control unit 107 controls the transmission unit 105 and the reception unit 106 to switch the condition from the first transmission/reception condition of the search mode to the second transmission/reception condition of the high-definition mode. By doing so, the search mode is switched to the high-definition mode for acquiring the 2-dimensional elastic frame data with the higher definition than the search mode. Also, the control unit 103 outputs the command to swing to the motor of the swinging jig (not shown) mounted in the probe 102 in accordance with the command for switching to the high-definition mode. By this means, as shown in FIG. 3(b), the ultrasonic scan surface of the probe 102 which is attached to, for example a swinging arm of the swinging jig is swung, and the swinging angle φ is controlled.

This allows the pressing operation to be stabilized as shown in time zone T3 of FIG. 3(c) and consecutively repeated, as well as the swinging angle φ of the ultrasonic scan surface to be changed within a set range. In this manner, the 2-dimensional elastic frame data of the high-definition mode in a certain 3-dimensional section which is centered around a region of interest can be consecutively acquired by the 2-dimensional elastic image constructing unit 116, and elastic volume data with high definition can be generated by the elastic volume data generation unit 117. In the case that the swinging angle φ is reciprocated within a set range, plural sets of high-definition elastic volume data of the 3-dimensional section including the region of interest will be generated. In FIG. 3(b), a pre-frame and a post-frame are a pair of RF frame data (Fr.0, Fr.1), . . . , (Fr.n−1, Fr.n) having different acquisition times, i.e. having different pressing amounts that are related to the calculation of the elastic frame data, and a set of 2-dimensional elastic frame data is acquired for each pair of RF frame data. Accordingly, elastic volume data of the high-definition mode can be acquired.

By performing rendering on the acquired high-definition elastic volume data, a 3-dimensional elastic image with high definition can be generated. FIG. 4 shows a processing flow in the ultrasonic diagnostic apparatus of the present embodiment up to switching from the search mode to the high-definition mode and creation of a high-definition 3-dimensional elastic image, by dividing the procedure into steps S1˜57.

FIG. 5 shows a modification example of FIG. 3. The difference in the embodiment shown in FIG. 5 from the embodiment shown in FIG. 3 is that the probe 102 is made swung in time zone T1 and time zone T2 of the search mode, and that the evaluation of stability and continuity is performed by prolonging time zone 2. In accordance with the present embodiment, since the elastic volume data is acquired by swinging the probe 102, a region of interest can be searched by observing the 3-dimensional elastic image created by rendering in real time. The definition of the 3-dimensional elastic image at this time is the rough mode by the first transmission/reception condition. The examiner can also set the length of time zone for making evaluation in the switching unit 121 by operating the operation unit 104. The switching unit 121 performs evaluation of the stability and continuity in the set time zone.

In other words, as shown in FIG. 5(a), a region of interest is searched by executing 3-dimensional scanning in real time and displaying the 3-dimensional image on the display unit 120. At this time, it is possible to evaluate the stability and continuity of variation in the elasticity value of the elastic volume data as shown in FIG. 5(c) by acquiring the variation pattern of the elasticity value at the time that the 2-dimensional elastic image is acquired. If the stability and continuity of variation is recognized in the elasticity value, determination can be made that a region of interest is captured and stable pressing operation has started. On the basis of this determination, acquisition of high-definition elastic volume data can be carried out by switching the search mode to the high-definition mode as in FIG. 3. In this manner, it is possible to obtain 3-dimensional elastic images with high definition that are appropriate for making diagnosis.

In accordance with the present embodiment, the ultrasonic diagnostic apparatus comprising transmission/reception processing units 105 and 106 configured to transmit/receive ultrasonic waves to/from an object 101 via a probe 102, a 2-dimensional elastic image constructing unit 116 configured to acquire elastic frame data showing the distribution of an elasticity value on the basis of the received ultrasonic signal and generate a 2-dimensional elastic image, a 3-dimensional elastic image constructing unit 118 configured to generate a 3-dimensional elastic image on the basis of plural sets of elastic frame data and a display unit 120 configured to display at least one of the 2-dimensional elastic image and the 3-dimensional elastic image, is further provided with a switching unit 121 configured to detect the variation of the elasticity value in plural sets of elastic frame data and switches the transmitting/receiving condition of an transmission/reception processing units 105 and 106 based on the variation in the elasticity value. The transmitting/receiving conditions of the transmission/reception processing units 105 and 106 are a first transmitting/receiving condition that acquires elastic frame data with a set first definition and a second transmitting/receiving condition that acquires elastic frame data with a second definition which is higher than the first definition, and the switching unit 121 loads the consecutive plural sets of elastic frame data acquired by the first transmitting/receiving condition from the 2-dimensional elastic image constructing unit and evaluates the stability of variation in the elasticity value. The method for transmitting and receiving ultrasonic signals includes a step of transmitting/receiving ultrasonic waves via the probe 102, a step of detecting variation of the elasticity value in plural sets of elastic frame data showing the distribution of the elasticity value on the basis of the received ultrasonic signals, and a step of switching the transmitting/receiving condition based on the stability of variation in the elasticity value.

Also, the display unit 120 can display the stability in variation of the elasticity value in plural sets of elastic frame data along with a 3-dimensional elastic image. Accordingly, an examiner can confirm whether the currently displayed 3-dimensional elastic image is generated in a stable condition and whether the 3-dimensional elastic image is generated by the first-definition mode or the second-definition mode, based on the stability of variation in the elasticity value.

### Embodiment 2

Other embodiments related to the switching unit 121 of the present invention will be described referring to FIG. 6. The difference from the first embodiment is that elastic volume data and 3-dimensional elastic images are generated using the pressure applied to biological tissue due to beats in place of the pressing operation by the probe 102. FIG. 6(a) is the operation mode for searching a region of interest as in the first embodiment, which searches a region of interest while acquiring two sets of RF signal frame data Fr.0 and Fr.1 with the frame rate of the first definition by moving the probe 102 with the fixed swinging angle φ and an arbitrary searching speed and observing the 2-dimensional elastic image displayed on the display unit 120. In this manner, the time graph of the elasticity value in biological tissue according to the intensity and cycles of the compression caused by beats can be obtained.

Here, when the pressure caused by beats is used, the variation pattern of the elasticity value is equivalent to, for example that of electrocardiographic complex. However in the search mode, there are times that the searching speed varies irregularly and the interval (pitch) between the two sets of RF frame data for acquiring elastic frame data varies irregularly. In this case, from the condition that the variation pattern of the elasticity value varies irregularly, the search mode can be automatically switched to the high-definition mode when the change to regular and stable variation pattern is detected. That is, when the examiner starts acquiring a high-definition 3-dimensional elastic image, the variation pattern of the elasticity value appears in the graph in which the displacement shows periodic, stable and large waves according to the peaks of the beats, as shown in FIG. 6(c). Given this factor, the switching unit 121 in the present embodiment detects the variation pattern of the elasticity value and evaluates the stability and continuity of the variation pattern, as in the first embodiment. Then by outputting the command for switching to the high-definition mode on the basis of the evaluation result, the mode is switched automatically to acquisition of high-definition elastic volume data.

In particular, the switching unit 121 of the present embodiment detects the peak in the elasticity value, outputs the command for switching to the high-definition mode in the timing of the peak in the elasticity value at the same time as transmitting the command to a swinging motor in the probe 102, and causes the swinging to stop during period ΔT which is necessary for acquiring at least two sets of 2-dimensional elastic frame data. Further, the switching unit 121 detects the cycle of the peak, and causes the probe 102 to intermittently swing at address positions (φ0˜φ1) of the swinging angle that are set in advance for each cycle. The spacing between the address positions is set as a certain swinging angle Δφ in accordance with the high-definition frame rate. In this manner, elastic volume data by high-definition mode can be collected in appropriate pressing condition by using the compression caused by beats, thereby high-definition 3-dimensional images can be generated.

### Embodiment 3

The third embodiment of the switching unit 121 will be described referring to FIG. 7. The present embodiment is the method, in the case that the stability of pressing operation is lowered in the middle of acquiring the high-definition 2-dimensional elastic frame data after being switched to the high-definition mode, capable of automatically reacquiring the high-definition elastic volume data. More specifically, as shown in FIG. 7(a), a pair of 2-dimensional tomographic frame data sets (Fr.0 and Fr.1) are acquired by the high-definition mode, and plural sets of 2-dimensional frame data are consecutively acquired with respect to a volume region including the region of interest while swinging the probe 102.

In this process, the variation of the elasticity value in the high-definition mode is calculated with time as shown in the left side of FIG. 7(d).

At this time, when the stability of variation in the elasticity value breaks down in the middle of data acquisition, the switching unit 121 of the present embodiment outputs the reset command to reset the high-definition switching command. In response to this, the control unit 103 outputs to the transmission/reception control unit 107 to reset the transmitting/receiving condition of the search mode. When the reset command is input, the transmission/reception control unit 107 returns the probe 102 to the starting position for acquiring the first 2-dimensional tomographic frame data sets (Fr.0, Fr.1), and switches to the transmission/reception condition of the search mode. Then the switching unit 121, when the stability and continuity of the pressing operation is detected, switches the operation again as shown in FIG. 7(c) to acquire 2-dimensional elastic frame data by the high-definition mode as in the first embodiment. In this manner, as shown in FIG. 7(d), elastic volume data can be acquired with high definition, thereby making it possible to generate 3-dimensional elastic images with high definition.

For example, if the determination is made that the stability of the pressing operation broke down during acquisition of the elastic volume data after the elastic volume data of a region of interest is acquired, all of the acquired elastic volume data will be wasted. As a result, the time spent on reacquiring the entire elastic volume data will also be wasted. In contrast, in accordance with the present embodiment, since deterioration of the stability in variation of the elasticity value is detected and the elastic volume data is re-acquired automatically, it is possible to constrain the increase of time for acquiring elastic volume data.

### Embodiment 4

The fourth embodiment of the switching unit 121 will be described referring to FIG. 8. In the first embodiment, the stability and continuity of pressing operation is evaluated based on the variation pattern of the elasticity value in a specified section of the plural sets of 2-dimensional elastic frame data acquired consecutively by the search mode. Then starting of elastic volume data acquisition is determined on the basis of the evaluation and the high-definition mode switching command is output, so as to acquire high-definition elastic volume data. In contrast, the present embodiment is different in that starting of elastic volume data acquisition is determined by evaluating the stability and continuity of pressing operation based on the elastic volume data of the rough search mode formed by the plural sets of 2-dimensional elastic frame data acquired by the search mode. In other words, the present embodiment is characterized in that the stability of variation in the elasticity value is evaluated by comparing the correlation value or the noise ratio between the adjacent two sets of 2-dimensional elastic frame data that forms elastic volume data and the set values thereof.

Here, it is effective to obtain the correlation value or the noise ratio between the adjacent two sets of 2-dimensional elastic frame data in the entire volume, calculate the average value in the volume, and compare the volume average value and the threshold value (set value) for evaluation. More specifically, the pressing operation can be evaluated as being stable when the volume average of the correlation value between two adjacent sets of adjacent 2-dimensional elastic frame data that form the elastic frame data is greater than a preset threshold value. Also, the pressing operation can be evaluated as being stable when the volume average of the noise ratio between two sets of adjacent 2-dimensional elastic frame data is smaller than a preset threshold value.

Given this factor, the correlation value of elastic volume data sets V0˜V4 acquired by reciprocating and swinging the probe 102 in the range of a region of interest as shown in FIG. 8(a) or volume average VQ of the noise ratio is obtained in real time as shown in FIG. 8(c). Then when volume average VQ is greater (or smaller) than the threshold value, the examiner determines that the acquisition of high-definition elastic volume data has started and outputs the high-definition mode switching command. In this manner, the transmitting/receiving condition is switched to the high-definition mode as shown in FIG. 8(b), and elastic volume data with high definition is acquired as shown in FIG. 8(c).

### Embodiment 5

The fifth embodiment of the switching unit 121 will be described referring to FIG. 9. The present embodiment is the modification of the fourth embodiment. As shown in FIGS. 9(a) and (b), plural sets of elastic volume data V0˜V4 by the search mode with rough definition formed by the plural sets of 2-dimensional elastic frame data acquired by the search mode is consecutively obtained. At this time, a 3-dimensional elastic image can be rendered based on the acquired plural sets of elastic volume data V0˜V4 and displayed on the display unit 120. Then the degree of similarity (for example, correlation function C) between two pairs of elastic frame data acquired at adjacent acquisition times (for example, V0 and v1, V1 and V2) is sequentially obtained. When the sequentially obtained degree of similarity (for example, similarity between V3 and V4) surpasses the preset threshold value, the pressing operation is evaluated as being stable so that the examiner determines that the acquisition of high-definition volume data has started and outputs the high-definition mode switching command. In this manner, the transmitting/receiving condition is switched to the high-definition mode as shown in FIG. 9(c), and elastic volume data with high definition can be acquired as shown in FIG. 9(b). Accordingly, the present embodiment is different in that the degree of similarity of the adjacent two pairs of elastic volume data is used in place of the correlation value or the noise ratio in the fourth embodiment.

### Embodiment 6

The sixth embodiment regarding the switching unit 121 will be described referring to FIG. 10. In the first˜fifth embodiments, the mode for acquiring the elastic volume data is automatically switched to the high-definition mode when stability is detected in variation of the elasticity value or when it is detected that the quality of the elastic volume data is greater than (or smaller than) the threshold value. In contrast, the sixth embodiment searches a region of interest by displaying on the display unit 120 a 2-dimensional tomographic image by the search mode or a 3-dimensional elastic image by performing pressing operation as in the fifth embodiment. In the present embodiment, when a region of interest is captured by the search mode, the high-definition switching command is manually input to the switching unit 121 from a device such as the operation unit 104. In this manner, the switching unit 121 displays preset waiting time Tw (for example, 10 seconds) on the display unit 120 and keeps the time, then outputs the high-definition switching command to the transmission/reception control unit 107 via the control unit 103 when Tw expires, for switching to the high-definition mode. In other words, in the present embodiment, the examiner manually inputs the high-definition switching mode via a device such as the operation unit 104 when the region of interest is captured. At this time, even when the scanning position of the probe 102 is displaced from the region of interest or the pressing operation becomes unstable, high-definition elastic volume data can be acquired by capturing the region of interest during waiting time Tw and performing stable pressing operation. As a result, it is possible to generate high-definition 3-dimensional elastic images as in the other embodiments.

### Embodiment 7

The seventh embodiment regarding the switching unit 121 will be described referring to FIG. 11. The present embodiment is characterized in that a 3-dimensional elastic image with rough definition and a 3-dimensional elastic image with high-definition are alternately obtained at regular time intervals and displayed on the display unit 120. More specifically, the switching unit 121 is reset after the command for switching to the high-definition mode is output at regular time intervals (for example, 60 seconds). In this manner, the transmission/reception control unit 107 controls the transmission unit 105 and the reception unit 106 by alternately switching the first transmitting/receiving condition and the second transmitting/receiving condition. As a result, the elastic volume data with rough definition is acquired by performing pressing operation by the search mode, and the 3-dimensional elastic image with rough definition is displayed on the display unit 102 in real time. Also, the elastic volume data with high definition is acquired by performing pressing operation by the high-definition mode, and the 3-dimensional elastic image with high definition is displayed on the display unit 120 in real time. In other words, the 3-dimensional elastic image by the search mode and the 3-dimensional elastic image by the high-definition mode in real time are alternately acquired and displayed. Therefore, the present embodiment is capable of searching a region of interest using the 3-dimensional elastic image by the search mode and observing the 3-dimensional elastic image by the high-definition mode in the next cycle.

In the present embodiment, the number of elastic volume data sets of the search mode and the high-definition mode do not have to be the same. For example, it is possible to acquire ten sets of elastic volume data by the search mode and one set of elastic volume data by the high-definition mode. The switching may also be performed, for example such that the volume data of the search mode is acquired for 30 seconds and the elastic volume data by the high-definition mode is acquired for one volume. Also as shown in FIG. 11(c), an indicator such as a progress bar can be displayed on the display unit 120 so that the time up to the next high-definition mode can be easily recognized. It is also preferable, at the time that the elastic volume data with high definition is acquired, to automatically store the acquired data as filing data or raw data.

FIG. 12 shows an example of a progress bar in the present embodiment to be displayed at the time of obtaining a high-definition 3-dimensional elastic image with waiting time Tw and by time schedule Ts. The progress bars shown in FIG. 12(a) indicate that the acquisition of a high-definition 3-dimensional elastic image has started in the timing that the hatched-line portion disappeared from the bar. Thus the acquisition of a high-definition 3-dimensional elastic image is to be started at the time that the indication in the progress bar reaches with time the bottom of the bars in the diagram in which the occupation rate of the hatched-line portion becomes zero. In addition, the progress bar can also be used for comparing and displaying the stability of variation in the elasticity value, the continuity of stability, or evaluation value of the quality of elastic volume data, etc. with the threshold value.

### DESCRIPTION OF REFERENCE NUMERALS

100 ultrasonic diagnostic apparatus
102 ultrasonic probe
103 control unit
104 operation unit
105 transmission unit
106 reception unit
107 transmission/reception control unit
108 phasing and adding unit
109 data storage unit
113 2-dimensional tomographic image constructing unit
114 tomographic volume data generating unit
115 3-dimensional tomographic image constructing unit
116 2-dimensional elastic image constructing unit
117 elastic volume data generating unit
118 3-dimensional elastic image constructing unit
119 synthesis processing unit
120 display unit
121 switching unit

1. An ultrasonic diagnostic apparatus comprising:
a transmission/reception processing unit configured to transmit/receive ultrasonic signals to/from an object to be examined via a probe;
a 2-dimensional elastic image constructing unit configured to acquire the elastic frame data showing the distribution of elasticity values on the basis of the received ultrasonic signals and generate a 2-dimensional elastic image;
a 3-dimensional elastic image constructing unit configured to generate a 3-dimensional elastic image based on plural sets of the elastic frame data; and
a display unit configured to display at least one of the 2-dimensional elastic image and the 3-dimensional elastic image,
further comprising a switching unit configured to detect variation of the elasticity value in the plural sets of elastic frame data and switches the transmitting/receiving condition of the transmission/reception processing unit based on the stability of variation in the elasticity value.

2. The ultrasonic diagnostic apparatus according to claim 1, wherein:
the transmitting/receiving condition of the transmission/reception processing unit is a first transmitting/receiving condition which acquires the elastic frame data with a set first definition and a second transmitting/receiving condition which acquires the elastic frame data with a second definition that is higher than the first definition; and
the switching unit loads the consecutive plural sets of elastic frame data acquired by the first transmitting/receiving condition from the 2-dimensional elastic image constructing unit and evaluates the stability of variation in the elasticity value.

3. The ultrasonic diagnostic apparatus according to claim 2, wherein the switching unit evaluates the stability of variation in the elasticity value based on the variation pattern of the detected elasticity value.

4. The ultrasonic diagnostic apparatus according to claim 3, wherein the switching unit, when the variation cycles have the variation pattern in which the elasticity value repeatedly increases and decreases, acquires the difference between the variation pattern feature quantities of two consecutive half cycles or full cycles in the variation cycles, and makes evaluation that the variation pattern is stable when the acquired difference is within a set range.

5. The ultrasonic diagnostic apparatus according to claim 4, wherein the switching unit switches the transmitting/receiving condition from the first condition to the second condition based on the continuity in which the stability of variation pattern continues for a set number of cycles.

6. The ultrasonic diagnostic apparatus according to claim 2, wherein the first or the second definition is set by at least one of the density in the transmitting/receiving beam of the ultrasonic signals and the frame rate.

7. The ultrasonic diagnostic apparatus according to claim 2, wherein the elasticity value is any one of the displacement, strain, elasticity modulus, viscosity of biological tissue, the strain ratio with respect to a reference area or other physical quantity which correlates with elasticity.

8. The ultrasonic diagnostic apparatus according to claim 2, further comprising an elastic volume data generating unit configured to collect plural sets of the elastic frame data acquired with the second definition by the 2-dimensional elastic image constructing unit and generates elastic volume data with high definition.

9. The ultrasonic diagnostic apparatus according to claim 3, wherein the switching unit, when stability of the variation pattern breaks down after being switched to the second transmitting/receiving condition, resets the first transmitting/receiving condition, then switches to the second transmitting/receiving condition again based on the evaluation of stability in the variation pattern.

10. The ultrasonic diagnostic apparatus according to claim 2, wherein:
the 2-dimensional elastic image constructing unit acquires the elastic frame data showing the distribution of the elasticity value in biological tissue that is deformed by receiving compression caused by beats; and
the switching unit loads consecutive plural sets of the elastic frame data, detects the peak in variation pattern of the elasticity value caused by the beats, and switches the transmitting/receiving condition of the transmission/reception processing unit based on the stability in the peak cycle.

11. The ultrasonic diagnostic apparatus according to claim 10, wherein:
the transducers of the probe are mounted to a jig which is swung by a motor in the direction that intersects with a tomographic plane; and
the switching unit outputs the signal to stop the motor for a set period of time when the peak is detected, and after passing of the set period of time, drives the motor to make the tomographic plane position of the transducers to swing by a set angle in accordance with the peak cycle.

12. The ultrasonic diagnostic apparatus according to claim 2, wherein the 3-dimensional elastic image constructing unit divides plural sets of elastic volume data respectively into plural frame blocks, creates one set of elastic volume data by combining the frame blocks of which the elasticity value is within a certain allowable range, and constructs a 3-dimensional elastic image by performing rendering on the created elastic volume data.

13. The ultrasonic diagnostic apparatus according to claim 2, further comprising an elastic volume data generating unit configured to collect plural sets of the elastic frame data acquired by the 2-dimensional elastic image constructing unit and generates elastic volume data, wherein the switching unit evaluates the stability of variation in the elasticity value by loading the elastic volume data acquired by the first transmitting/receiving condition from the elastic volume data generating unit and comparing the correlation values between two adjacent sets of elastic frame data that form the elastic volume data or the noise ratio with the set values thereof.

14. An ultrasonic diagnostic apparatus comprising:
a transmission/reception processing unit configured to scan an ultrasonic beam to a tomographic plane of an object via a probe, receive the ultrasonic signal from the tomographic plane and generate a wave-receiving beam signal;
a 2-dimensional elastic image constructing unit configured to acquire the elastic frame data showing the distribution of the elasticity value of biological tissue in the tomographic plane on the basis of the wave-receiving beam signal and generate a 2-dimensional elastic image;
an elastic volume data generating unit configured to collect the elastic frame data with respect to plural tomographic planes of which the positions in the direction that intersects with the tomographic plane are different, and generate elastic volume data;
a 3-dimensional elastic image constructing unit configured to perform rendering on the elastic volume data and generate a 3-dimensional elastic image; and
a display unit configured to display at least the 3-dimensional elastic image,
wherein the transmission/reception processing unit is configured switchable between a first transmitting/receiving condition which acquires the elastic frame data with a set first definition and a second transmitting/receiving condition which acquires the elastic frame data with a second definition that is higher than the first definition,
further comprising a switching unit configured to alternately switch the first and the second transmitting/receiving conditions of the transmission/reception processing unit at every set interval.

15. An ultrasonic transmission/reception method including steps of:
transmitting/receiving ultrasonic signals via a probe;
detecting variation of the elasticity value in plural sets of elastic frame data showing the distribution of the elasticity value based on the received ultrasonic signal; and
switching the transmitting/receiving condition based on the stability in variation of the elasticity value.
